(19) United States
(12) Patent Application Publication
(10) Pub. No.: US 2017/0231996 A1
(43) Pub. Date: Aug. 17, 2017

(54) STABLE EDIBLE SUPERSATURATED CAFFEINE SOLUTIUONS AND METHODS OF MAKING THE SAME

(71) Applicant: INSPIRED BRANDS INTERNATIONAL, LLC, Weston, CT (US)

(72) Inventors: Robert Levi, Weston, CT (US); Steve Leusner, Chads Ford, PA (US)

(73) Assignee: INSPIRED BRANDS INTERNATIONAL, LLC, Weston, CT (US)

(21) Appl. No.: 15/289,922

(22) Filed: Oct. 10, 2016

Related U.S. Application Data
(60) Provisional application No. 62/239,144, filed on Oct. 8, 2015.

(51) Int. Cl. A61K 31/522; A23L 2/52; A61K 47/12; A61K 47/10; A61K 47/40; A61K 9/00

(52) U.S. Cl. CPC A61K 31/522; A61K 47/40; A61K 9/0095; A61K 9/0053; A61K 47/12; A61K 47/10; A23L 2/52; A23V 2002/00

(57) ABSTRACT

The present disclosure is generally directed to energy drinks, specifically, the present disclosure is directed to energy drinks having supersaturated caffeine and low relative sourness at the consumption and/or storage conditions.

### BACKGROUND

The present disclosure generally relates to energy drinks, specifically, the present disclosure relates to energy drinks having caffeine in an amount greater than the temperature-corresponding solubility limit.

There exist a number of drinks designed to aid human performance. However, the human performance drinks can be divided to three types: (1) those that provide electrolytes; (2) those that provide vitamins; and (3) those that provide energy through use of sugar and caffeine, and 4) those that provide caffeine and vitamins

Examples of energy drinks providing caffeine and sugar are, for example Red Bull® (Red Bull GmbH, Austria, with 83 mg caffeine in 238 ml), Monster® (Monster Beverage Corporation, Corona, Calif., with 92 mg caffeine in 227 ml.), Rockstar® (Rockstar Beverage Corporation, Lax Vegas, Nev. with about 80 mg caffeine in 227 ml),

Even when considering the high-energy shots, the amount of caffeine incorporated is limited by the solubility of caffeine in the liquid composition, and the bitter taste resulting at these high concentrations. For example, Clif Bar & Co.'s Clif Shot Turbo Double Espresso Energy Gel provides about 133 mg caffeine in about 55 ml, while 5-Hour Energy® (Extra Strength, Living Essentials, Wabash, Ind., with about 242 mg caffeine in 56 ml. Similarly, Rockstar® (Shots, Rockstar Beverage Corporation, Lax Vegas, Nev.) provide about 229 mg caffeine in 70 ml.

In the art, the energy drinks are used to overcome tiredness due to lack of sleep and/or lack of sleep by increasing heart rate and raising sugar levels in the body. Under certain circumstances, for portability, being discrete and general convenience, it would be advantageous to obtain a shot having higher amount of caffeine, in a smaller serving size

### SUMMARY

The present disclosure provides embodiments of energy drinks or energy shots solution. Specifically, provided herein are embodiments of edible solutions of caffeine having caffeine concentration above the solubility limit at room temperature, while remaining stable from recrystallization (and therefore precipitation from the solution) for a period of no less than 6 weeks, while maintaining acceptable flavor profile with low sourness.

In an embodiment, provided herein is a supersaturated aqueous solution of caffeine having a caffeine concentration of between about 1.7% (w/v) and about 3.0% (w/v), a caffeine isomer having a concentration of between about 0.05% (w/v) and about 0.10% (w/v), an organic acid, cyclodextrine (CD) and water, the supersaturated solution is stable at room temperature for a period of no less than about 12 weeks.

In another embodiment, provided herein is a method of preventing caffeine recrystallization from a solution at temperature range of between about 4° C. and about 25° C., the caffeine is present at a supersaturation ratio of between about 1.1 and about 1.7, the method comprising: combining a glycol and/or glycerin with water at a ratio of glycol and/or glycerin to water between about 1:20 and about 1:5; admixing caffeine at a supersaturation level of between about 1.1 and about 1.7 solution temperature of about 22° C.; admixing a caffeine isomer and a cyclodextrine, wherein the ratio of the caffeine to caffeine isomer of between about 17 and 60; and admixing organic acid at a concentration sufficient to neutralize between about 25% and about 35% of the caffeine.

### DETAILED DESCRIPTION

Provided are embodiments of edible supersaturated solutions of caffeine having caffeine concentration above the solubility limit at room temperature, while remaining stable from recrystallization (and therefore precipitation from the solution) for a period of no less than 6 weeks at temperatures between about 25° C. and about 4.0° C.

Caffeine (1,3,7-Trimethylpurine-2,6-dione):

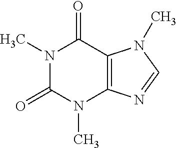

Illustrated above, Caffeine (1,3,7-Trimethylpurine-2,6-dione) can be soluble in water at room temperature at a level of 1.6%-2.0 (w/v, g/ml). The solubility of Caffeine goes up dramatically in hot water (e.g., ˜67% in boiling water) and down drastically in cold water, for example, to 0.6 g/100 ml water at 0° C., or 1 g/100 ml in water at 15° C.

Below the temperature-dependent solubility limit, the solution can become super-saturated, and the caffeine may recrystallize and precipitate out of solution. Crystallization kinetics can typically be characterized by the onset time required for measurable crystal nucleation and growth to occur (e.g., induction time). The induction time can be dependent on a number of parameters including temperature, concentration, solvent system, and fluid dynamics, as well as interfacial tension between the solution and the vessel in which it is contained. For caffeine, high interfacial surface tension, low temperatures, low viscosity and high concentration will reduce the induction time and increase the rate of recrystallization. Moreover, ethanol reduces caffeine solubility, which makes the introduction of caffeine into alcoholic beverages more challenging.

The compositions and methods provided herein can be configured to provide edible caffeine solutions, emulsions, suspensions, gels and a combination comprising one or more of the foregoing and other suspended systems, some shock tolerance to cold temperatures, and complete stability at room temperature. The level of caffeine provided can be between about 150 mg caffeine in 9 ml (g) water and 250 mg/9 ml (g) or beyond, or between about 1.66% (w/v) and about 3% (w/v). The compositions described can therefore deliver stable caffeine in suspended systems has supersaturation ratio (relative to water) of between about 1.1 and about 1.7. Additionally, the compositions described and claimed herein can be configured to mask the taste of caffeine at these supersaturation levels, which can be very bitter and lingering. Not wishing to be bound by theory, the masking effect can be due to conversion to (organic/mineral acid) salt form of caffeine as opposed to base form.

The term “supersaturated,” as used herein, means having a compound in a solvent in which it is completely dissolved at a certain temperature but at which the solubility of the compound in the solvent at that certain temperature is exceeded. In other words, the term “supersaturated” means that the preparations, compositions, combinations, solutions, or any other suspended systems of caffeine described herein are storage-stable and free of precipitation or creaming of caffeine in spite of the high concentration of caffeine. The term “supersaturated solution” as used herein has the same meaning generally understood in the art, namely a homogeneous solution which contains more of a solute (an excess) than is normally possible at a given temperature. Likewise, the term “supersaturation ratio” refers in an embodiment to the ratio: (concentration of caffeine in the formulation at the storage and/or consumption conditions/concentration of caffeine soluble in water at the storage and/or consumption conditions).

Accordingly and in an embodiment, provided herein is a supersaturated aqueous solution of caffeine having a caffeine concentration of between about 1.7% (w/v) and about 3.0% (w/v), a caffeine isomer having a concentration of between about 0.05% (w/v) and about 0.10% (w/v), an organic acid, cyclodextrine (CD) and water, wherein the solution can be stable at temperature over about 4° C. for a period of no less than about 6 weeks, or in another embodiment, for a period of no less than 12 weeks at a temperature of 10° C.

The term stable, refers in an embodiment to describe a solution containing supersaturated levels of caffeine, wherein the supersaturation is between about 1.1 and about 1.7 at 4° C., having induction times (in other words, the onset time required for measurable crystal nucleation and growth to occur) over the stated stability time (e.g., 6 weeks).

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

The glycol used in the compositions of aqueous solution of supersaturated caffeine concentration and methods for its stabilization can be, propylene glycol, propylene glycol diester, glycerin, 1,3 propanediol, or combination comprising one or more of the foregoing or combination comprising one or more of the foregoing. Likewise, the ratio between the glycol, 1,3 propanediol and/or glycerin, and water used in the compositions of aqueous solution of supersaturated caffeine concentration and methods for its stabilization can be between about 1:20 and about 1:5. The glycol, diol, and/or glycerin, can be used in certain embodiments, to mask the bitter flavor of the supersaturated caffeine solutions, or the taste resulting from the salt formed through the neutralization of the caffeine (a weak base having pKₐ of 0.6) using organic acid. In addition, depending on the glycol and/or glycerin used, the viscosity of the edible solution can be increased somewhat, thus further increasing induction times, by, for example, delaying caffeine self-diffusion that can be necessary for recrystallization.

For example, the glycol, diol, and/or glycerin used in the compositions of aqueous solution of supersaturated caffeine concentration and methods for its stabilization can be propylene glycol at a ratio of propylene glycol to water of 1:9.

Furthermore, the organic acid used in the compositions of aqueous solution of supersaturated caffeine concentration and methods for its stabilization can be, for example, ascorbic acid, citric acid, fumaric acid, glutaric acid, lactic acid, malic acid, sorbic acid, tartaric acid or a combination comprising one or more of the foregoing. Specifically, the organic acids, which, with other components of the solution (e.g., sweeteners), will produce the desired solubilizing effect in the present disclosure, can be one or more of those selected from the group consisting of adipic, ascorbic, citric, fumaric, lactic, malic and tartaric acids. These organic acids occur in natural products, although, as a practical matter, they may be synthesized for low-cost mass production. Adipic acid occurs in beet juice. Ascorbic acid is found in citrus fruits, hip berries, and fresh tea leaves. Citric acid is present in lemon, lime and grapefruit juices, to name a few sources Fumaric acid is found in many plants. Lactic acid makes sour milk tart. Malic acid is found in unripe fruits, maple juice and apples. Finally, tartaric acid occurs in fruit and vegetable tissues. The choice of organic acid, or combination of organic acids used in the compositions of aqueous solution of supersaturated caffeine concentration and methods for its stabilization can be determined based on other flavor and other components of the edible solutions described and claimed. Mineral acids such as hydrochloric or phosphoric may also be used.

In an embodiment, the organic acid used in the compositions of aqueous solution of supersaturated caffeine concentration and methods for its stabilization can be present in the solution at a concentration sufficient to neutralize no less than about 25% (w/w) of the caffeine, or in another embodiment, between about 27% (w/w) and 40% of the caffeine. For example, the organic acid can be citric acid (but it will be understood that any one, or combination of the other organic acids listed may be substituted with the same results), present in the solution at a concentration sufficient to neutralize between about 25% and about 35% of the caffeine.

The stable, supersaturated caffeine solutions, emulsions, gels and the like suspended systems provided herein can be formulated to have reduced sourness, resulting from the organic acid used in the composition. In an embodiment, reducing sourness can be done by producing double emulsion of water-in-oil-in-water (W/O/W), where the neutralized caffeine, the caffeine isomer, and the cyclodextrin are all incorporated in the internal phase, emulsified in oil and (W/O) and then reemulsified in water to yield the duplex emulsion.

The solutions described herein can also be configured to be stable at a temperature of no less than 10° C. for a period of no less than 12 weeks, or in other words, have induction time for caffeine recrystallization of no less than 12 weeks at storage or consumption temperature of 10° C. (50° F.).

The aqueous solution of high concentration caffeine having supersaturated caffeine concentration at the storage and/or consumption temperature, can further comprise The compositions of the present invention may further comprise additional optional components to enhance, for example, their performance in providing energy, mental alertness, organoleptic properties, and nutritional profile. For example, one or more, flavanols, acidulants, coloring agents, minerals, soluble fibers, non-caloric sweeteners, flavoring agents, preservatives, emulsifiers, oils, carbonation components, and the like may be included in the compositions herein. Such optional components may be dispersed, solubilized, or otherwise mixed into the compositions described and claimed. These components may be added to the compositions herein provided they do not substantially hinder the properties of the beverage composition, particularly the induction time for supersaturated caffeine recrystallization. Non-limiting examples of optional components suitable for use herein are given below.

An optional, but particularly preferable component of the present invention is one or more plant phytochemical constituents. This would include flavanols or other phytochemicals which are in essence “healthy.” The inclusion of one or more flavanols serves to delay the glycemic response associated with ingestion of the present compositions, thus providing further maintenance of energy to the user. Because one or more flavanols will contribute to the onset, and particularly maintenance of energy wherein the composition is ingested, it is particularly preferred that one or more flavanols be included.

The aqueous solution, emulsions, suspensions, gels and a combination comprising one or more of the foregoing and other suspended systems, of high concentration caffeine having supersaturated caffeine concentration at the storage and/or consumption temperature may optionally further comprise one or more acidulants. An amount of an acidulant may be used to maintain the pH of the composition. Compositions of the present invention preferably have a pH of from about 2 to about 8, for example, from about 2 to about 5, or from about 2 to about 4.5, specifically from about 2.7 to about 4.2. Beverage acidity can be adjusted to and maintained within the requisite range by known and conventional methods, e.g., the use of one or more acidulants. Typically, acidity within the above recited ranges is a balance between maximum acidity for microbial inhibition and optimum acidity for the desired flavor.

Small amounts of one or more coloring agents may be utilized in the aqueous solution, emulsions, suspensions, gels and a combination comprising one or more of the foregoing and other suspended systems, of high concentration caffeine having supersaturated caffeine concentration at the storage and/or consumption temperature described. β-carotene can also be used. Riboflavin and FD&C dyes (e.g., yellow #5, blue #2, red #40) and/or FD&C lakes may also be used. By adding the lakes to the other powdered ingredients, all the particles, in particular the colored iron compound, are completely and uniformly colored and a uniformly colored beverage mix is attained. Additionally, a mixture of FD&C dyes or a FD&C lake dye in combination with other conventional food and food colorants may also be used. Additionally, other natural coloring agents may be utilized including, for example, chlorophylls and chlorophyllins, as well as fruit, vegetable, and/or plant extracts such as grape, black currant, aronia, carrot, beetroot, red cabbage, and hibiscus. Natural colorants are preferred for “all natural” drink products.

One or more soluble fibers may also optionally be included in the aqueous solution, emulsions, suspensions, gels and a combination comprising one or more of the foregoing and other suspended systems, of high concentration caffeine having supersaturated caffeine concentration at the storage and/or consumption temperature provided, for example, for satiation and refreshment, and/or nutritive benefits as well as for improving viscosity and mouthfeel. Soluble dietary fibers are a form of carbohydrates which cannot be metabolized by the enzyme system produced by the human body and which pass through the small intestine without being hydrolyzed.

Soluble fibers which can be used singularly or in combination in the aqueous solution of high concentration caffeine having supersaturated caffeine concentration at the storage and/or consumption temperature, include but are not limited to pectins, psyllium, guar gum, xanthan gum, alginates, gum arabic, inulin, agar, and carrageenan.

In addition, effective levels of non-caloric sweeteners may also optionally be used, in the edible compositions of supersaturated caffeine described and claimed to enhance the organoleptic and sweetness quality of the compositions. Non-limiting examples of non-caloric sweeteners include aspartame, saccharine, cyclamates, acesulfame K, L-aspartyl-L-phenylalanine lower alkyl ester sweeteners, L-aspartyl-D-alanine amides, L-aspartyl-D-serine amides, L-aspartyl-hydroxymethyl alkane amide sweeteners, L-aspartyl-l-hydroxyethylalkane amide sweeteners, glycyrrhizins, and synthetic alkoxy aromatics.

One or more flavoring agents can be used in the edible compositions of supersaturated caffeine described and claimed, in order to enhance their palatability. Any natural or synthetic flavor agent can be used. For example, one or more botanical and/or fruit flavors may be utilized herein. As used herein, such flavors may be synthetic or natural flavors.

The flavor agent can also comprise a blend of various flavors. If desired, the flavor in the flavoring agent may be formed into emulsion droplets which are then dispersed in the beverage composition or concentrate. Because these droplets usually have a specific gravity less than that of water and would therefore form a separate phase, weighting agents (which can also act as clouding agents) can be used to keep the emulsion droplets dispersed in the beverage composition or concentrate. Examples of such weighting agents are brominated vegetable oils (BVO) and resin esters, in particular the ester gums. See L. F. Green, Developments in Soft Drinks Technology, Vol. 1, Applied Science Publishers Ltd., pp. 87-93 (1978) for a further description of the use of weighting and clouding agents in liquid beverages. Typically the flavoring agents are conventionally available as concentrates or extracts or in the form of synthetically produced flavoring esters, alcohols, aldehydes, terpenes, sesquiterpenes, and the like.

Optionally, one or more preservatives may additionally be utilized herein. Preferred preservatives include, for example, sorbate, benzoate, and polyphosphate preservatives. Preferably, wherein a preservative is utilized herein, one or more sorbate or benzoate preservatives (or mixtures thereof) are utilized. Sorbate and benzoate preservatives suitable for use in the present invention include sorbic acid, benzoic acid, and salts thereof, including (but not limited to) calcium sorbate, sodium sorbate, potassium sorbate, calcium benzoate, sodium benzoate, potassium benzoate, and mixtures thereof. Natural preservatives are preferred such as those made by culturing dextrose.

In an embodiment, the aqueous solution, emulsions, suspensions, gels and a combination comprising one or more of the foregoing and other suspended systems, of high concentration caffeine having supersaturated caffeine concentration at the storage and/or consumption temperature has between about 170 mg and about 280 mg caffeine in a 9 ml solution, emulsions, suspensions, gel and a combination comprising one or more of the foregoing and other suspended systems, of high concentration caffeine having supersaturated caffeine concentration at the storage and/or consumption temperature.

In an embodiment, the aqueous solution, emulsions, suspensions, gels and a combination comprising one or more of the foregoing and other suspended systems, of high concentration caffeine having supersaturated caffeine concentration at the storage and/or consumption temperature described and claimed are formed using the methods described herein. Accordingly, in an embodiment, provided herein is a method of preventing caffeine recrystallization from a solution at temperatures below 22° C., the caffeine is present at supersaturated ratio between 1.1 and 1.7, the method comprising: combining a glycol and/or glycerin, with water at a ratio of glycol to water between about 1:20 and about 1:5; admixing caffeine at a supersaturation level of between about 1.1 and about 1.7 solution temperature of about 22° C.; and admixing organic acid at a concentration sufficient to neutralize no less than about 80% of the caffeine.

The supersaturated aqueous solutions of caffeine described herein can further comprise caffeine isomer, used in an embodiment to “poison” the crystal forming tendency of the supersaturated caffeine. The caffeine isomer used in the supersaturated suspended systems provided herein can be paraxanthine:

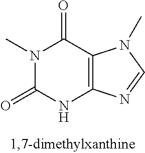

theobromine:

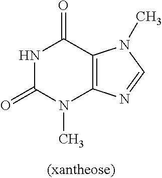

theacrine:

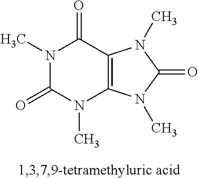 [SMILES: Cn1c(=O)c2c(n(C)c1=O)n(C)c(=O)n2C]

, or a caffeine isomer composition comprising the foregoing. The ratio between the caffeine and the caffeine isomer can be, for example, between 17 and 60, allowing a substantial reduction in the amount of organic acid used in the mixtures and compositions described.

The compositions further comprise cyclodextrines and/or cyclodextrine derivatives. Examples of cyclodextrins (the term cyclodextrin is hereinafter referred to as “CD”) used in the supersaturated aqueous solutions of caffeine described herein comprise α-CD, β-CD, γ-CD, δ-CD, ε-CD, polymer-CD and CD sugar. In an embodiment, the CD is γ-CD. The term “derivatives” used in conjunction with the term cyclodextrin refers to compounds in which at least one atom selected from hydrogen, oxygen or carbon in the cyclodextrin molecule is replaced by an atom or a group of atoms ordinarily present as a substituent in this kind of organic compounds (saccharides). These derivatives include etherified cyclodextrins, branched cyclodextrins, acylated cyclodextrins and sulfur-containing cyclodextrins. For example, etherified cyclodextrins can be (lower)alkylcyclodextrins such as methylcyclodextrin, ethylcyclodextrin, propylcyclodextrin, dimethylcyclodextrin, trimethylcyclodextrin etc., (lower)alkenylcyclodextrins, hydroxy(lower)alkylcyclodextrins such as hydroxyethylcyclodextrin, hydroxypropylcyclodextrin etc., (lower)alkoxy(lower)alkylcyclodextrins, aralkylcyclodextrins such as benzylcyclodextrin etc., halo(lower)alkylcyclodextrins such as chloroethylcyclodextrin etc., and cylodextrinepichlorohydrine copolymer and so on. These may be etherified cyclodextrins in which one, two or three hydroxy groups in any of the glucose units of the cyclodextrin molecule are converted into ether. Likewise, branched cyclodextrins include glucosylcyclodextrin, maltosylcyclodextrin etc; while acylated cyclodextrins include (lower)alkanoylcyclodextrins such as formylcyclodextin, acetylcyclodextrin etc., aromatically or heterocyclically acylated cyclodextrins such as benzoylcyclodextrin, nicotinoylcyclodextrin etc. Furthermore, sulfur-containing cyclodextrins include sulfonated cyclodextrins etc.

In an embodiment, CD (e.g., γ-CD), represented by the structure:

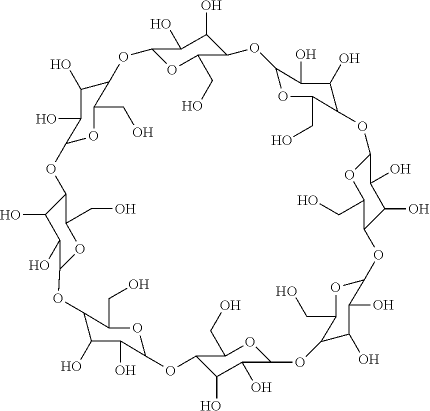 [SMILES: OCC1OC2OC3C(CO)OC(OC4C(CO)OC(OC5C(CO)OC(OC6C(CO)OC(OC7C(CO)OC(OC8C(CO)OC(OC9C(CO)OC(OC1C(O)C2O)C(O)C9O)C(O)C8O)C(O)C7O)C(O)C6O)C(O)C5O)C(O)C4O)C(O)C3O]

is present in the compositions described in a concentration of between about 1.10% and about 1.50%, for example, at a concentration of between about 1.20% (w/v) and about 1.30% (w/v).

The present invention is further illustrated with reference to the following nonlimiting examples.

### EXAMPLE 1

A) Caffeine is a weak base (pKa=0.6) with an equivalent wt. of 64.73 g/eq. Equivalents of citric acid, were added, converting the caffeine to a citric salt, which proved very soluble. 2.5 equivalents of citric acid to achieve room temperature solubility.
B) This however imparts a sour taste. Conversely, it also removes the bitterness of the caffeine
C) In order to reduce sourness, the stable salt solution was combined with the optimal level of propylene glycol allowing 1 eq. of citric acid to provide room temperature stability when in a solvent system of 90% (v/v) water and 10% (v/v) propylene glycol.
D) This stable caffeine base was also more easily flavored.

### EXAMPLE 2

A) To reduce sourness of the solution, Caffeine (natural) and citric acid were admixed at a ratio of ˜2.4/1.
B) γ-CD and theobromine at a ratio of 16.25 γ-CD/theobromine were added, where the ratio between caffeine (natural) and theobromine was 25 (w/w).
C) This removes the bitterness of the caffeine
D) In order to further reduce sourness, the stable salt solution was combined with the optimal level of propylene glycol allowing the citric acid to provide room temperature stability when in a solvent system of 90% (v/v) water and 10% (v/v) propylene glycol.
E) This stable caffeine base was also more easily flavored

The foregoing description is illustrative of particular embodiments of the invention, but is not meant to be a limitation upon the practice thereof.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All ranges disclosed herein are inclusive of the endpoints, and the endpoints are independently combinable with each other. “Combination” is inclusive of blends, mixtures, alloys, reaction products, and the like. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., the isomer(s) includes one or more isomers). Reference throughout the specification to “one embodiment”, “another embodiment”, “an embodiment”, and so forth, when present, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

Likewise, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such.

In an embodiment, the compositions provided are used in the methods described. Accordingly and in another embodiment provided herein is a method of preventing caffeine recrystallization from a solution at temperature range of between about 4° C. and about 25° C., the caffeine is present at a supersaturation ratio of between about 1.1 and about 1.7, the method comprising: combining a glycol and/or glycerin with water at a ratio of glycol and/or glycerin to water between about 1:20 and about 1:5; admixing caffeine at a supersaturation level of between about 1.1 and about 1.7 solution temperature of about 22° C.; admixing a caffeine isomer and a cyclodextrine, wherein the ratio of the caffeine to caffeine isomer of between about 17 and 60; and admixing organic acid at a concentration sufficient to neutralize between about 25% and about 35% of the caffeine.

The term “admix” or “admixture” refers to a combination of elements. For example, an admix of caffeine can refer to mixing 2 or more caffeine and its isomers together to form a mixture and is intended to encompass operations such as mixing, blending, combining and the like. It is possible to further define the admixture by indicating the percentage of one or more of the elements.

While particular embodiments have been described, alternatives, modifications, variations, improvements, and substantial equivalents that are or may be presently unforeseen may arise to applicants or others skilled in the art. Accordingly, the appended claims as filed and as they may be amended, are intended to embrace all such alternatives, modifications variations, improvements, and substantial equivalents.

1. A supersaturated aqueous solution of caffeine having a caffeine concentration of between about 1.7% (w/v) and about 3.0% (w/v), a caffeine isomer having a concentration of between about 0.05% (w/v) and about 0.10% (w/v), an organic acid, cyclodextrine (CD) and water, the solution is stable at room temperature for a period of no less than about 12 weeks.

2. The supersaturated aqueous solution of claim 1, wherein the glycol and/or glycerin, is, propylene glycol monoester, propylene glycol diester, glycerin, or combination comprising one or more of the foregoing.

3. The supersaturated aqueous solution of claim 2, wherein the ratio between the glycol and/or glycerin and water is between about 1:20 and about 1:5.

4. The supersaturated aqueous solution of claim 3, wherein the glycol and/or glycerin is propylene glycol at a ratio to water of 1:9.

5. The supersaturated aqueous solution of claim 4, wherein the organic acid is ascorbic acid, citric acid, fumaric acid, glutaric acid, lactic acid, malic acid, sorbic acid, tartaric acid or a combination comprising one or more of the foregoing.

6. The supersaturated aqueous solution of claim 5, wherein the organic acid is present in the solution at a concentration sufficient to neutralize no less than about 30% of the caffeine.

7. The supersaturated aqueous solution of claim 6, wherein the organic acid is citric acid, present in the solution at a concentration sufficient to neutralize between about 30% and about 40% of the caffeine.

8. The supersaturated aqueous solution of claim 7, wherein the cyclodextrine is present at a concentration of between about 1.10% and about 1.50%.

9. The supersaturated solution of claim 8, wherein the cyclodextrine is α-CD, β-CD, γ-CD, δ-CD, ε-CD, polymer-CD, CD sugar, or a CD composition comprising one or more of the foregoing.

10. The supersaturated aqueous solution of claim 9, wherein the CD is g-CD at a concentration of about 1.30%.

11. The supersaturated aqueous solution of claim 10, wherein the caffeine isomer is paraxanthine, theobromine, theacrine, or a caffeine isomer composition comprising the foregoing.

12. The supersaturated aqueous solution of claim 11, wherein the solution is stable at a temperature of no less than 10° C. for a period of no less than 12 weeks.

13. The supersaturated aqueous solution of claim 12, wherein the solution further comprises a flavoring agent, a thickening agent, a preservative, or a combination comprising one or more of the foregoing.

14. The supersaturated aqueous solution of claim 1, wherein the supersaturated solution has at least 200 mg caffeine in a 9 ml solution.

15. A method of preventing caffeine recrystallization from a solution at temperature range of between about 4° C. and about 25° C., the caffeine is present at a supersaturation ratio of between about 1.1 and about 1.7, the method comprising:
a. combining a glycol and/or glycerin with water at a ratio of glycol and/or glycerin to water between about 1:20 and about 1:5;
b. admixing caffeine at a supersaturation level of between about 1.1 and about 1.7 solution temperature of about 22° C.;
c. admixing a caffeine isomer and a cyclodextrine, wherein the ratio of the caffeine to caffeine isomer of between about 17 and 60; and
d. admixing organic acid at a concentration sufficient to neutralize between about 25% and about 35% of the caffeine.

16. The method of claim 15, wherein the glycol and/or glycerin is propylene glycol monoester, propylene glycol diester, glycerin, or combination comprising one or more of the foregoing.

17. The method of claim 16, wherein the cyclodextrine is α-CD, β-CD, γ-CD, δ-CD, ε-CD, polymer-CD, CD sugar, or a CD composition comprising one or more of the foregoing.

18. The method of claim 17, wherein the organic acid is citric acid, present in the solution at a concentration sufficient to neutralize between about 25% and about 35% of the caffeine.

19. The method of claim 18, wherein the caffeine isomer is paraxanthine, theobromine, theacrine, or a caffeine isomer composition comprising the foregoing.

20. The method of claim 11, wherein the solution has at least 200 mg caffeine in a 9 ml solution.
